(19) United States
(12) Patent Application Publication
(10) Pub. No.: US 2022/0031659 A1
(43) Pub. Date: Feb. 3, 2022

(54) NEUROPROTECTIVE COMPOUNDS AND METHODS OF USE

(71) Applicant: ARIZONA BOARD OF REGENTS ON BEHALF OF THE UNIVERSITY OF ARIZONA, Tucson, AZ (US)

(72) Inventors: Jon T. Njardarson, Tucson, AZ (US); Isaac Chogii, Tucson, AZ (US); David Townsend Smith, Tucson, AZ (US); Edon Vitaku, Chicago, IL (US); Daniela C. Zarnescu, Tucson, AZ (US); Rachel Allen, Tucson, AZ (US)

(73) Assignee: ARIZONA BOARD OF REGENTS ON BEHALF OF THE UNIVERSITY OF ARIZONA, Tucson, AZ (US)

(21) Appl. No.: 17/503,961

(22) Filed: Oct. 18, 2021

Related U.S. Application Data
(63) Continuation-in-part of application No. 16/515,743, filed on Jul. 18, 2019, now Pat. No. 11,147,795.
Application No. 16/515,743 is a continuation-in-part of application No. 15/329,615, filed on Jan. 27, 2017, now Pat. No. 10,358,438.
(60) Provisional application No. 62/714,256, filed on Aug. 3, 2018.
(60) Provisional application No. 62/027,209, filed on Jul. 21, 2014.
(60) Provisional application No. 62/038,143, filed on Aug. 15, 2014.
(60) Provisional application No. 62/131,948, filed on Mar. 12, 2015.

(51) Int. Cl. A61K 31/404; A61K 31/428; A61P 25/28

(52) U.S. Cl. CPC A61K 31/404; A61K 45/06; A61P 25/28; A61K 31/428

(57) ABSTRACT

The present disclosure relates to methods treating a clinical condition associated with a neurodegenerative disease or locomotor dysfunction in a subject. In particular, methods include administering to the subject in need of such a treatment a therapeutically effective amount of a compound of the formula:

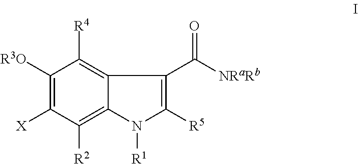

or a pharmaceutically acceptable salt thereof, or a prodrug thereof, where X, R^{a}, R^{b}, R¹, R², R³, R⁴, and R⁵ are those defined herein.

### CROSS-REFERENCE TO RELATED APPLICATIONS

This application is a continuation-in-part of U.S. patent application Ser. No. 16/515,743, filed Jul. 18, 2019, which is a continuation-in-part of U.S. patent application Ser. No. 15/329,615, filed Jan. 27, 2017, which is a U.S. National Stage Patent Application of PCT Patent Application No. PCT/US15/41169, filed Jul. 20, 2015, which claims the priority benefit of U.S. Provisional Application Nos. 62/027,209, filed Jul. 21, 2014; 62/038,143, filed Aug. 15, 2014; and 62/131,948, filed Mar. 12, 2015. This application also claims the priority benefit of U.S. Provisional Application No. 62/714,256, filed Aug. 3, 2018. All of these applications are incorporated herein by reference in their entirety.

### FIELD OF THE INVENTION

The present disclosure relates to methods treating a clinical condition associated with a neurodegenerative disease or locomotor dysfunction in a subject.

### BACKGROUND OF THE INVENTION

Amyotrophic Lateral Sclerosis (ALS) is an adult onset, progressive neurological disorder characterized by selective degeneration and death of motor neurons in the motor cortex and the spinal cord. Approximately 10% of all ALS cases are inherited and have been linked to a number of genes including superoxide dismutase (SOD1) and more recently C9ORF72 among others. However, 90% of the known ALS cases are sporadic and remain poorly understood. The ALS pathology includes ubiquitin positive cytoplasmic bodies, which have been shown to contain a 28 kDa fragment corresponding to the C-terminus domain of TDP-43 protein together with the full length TDP-43. TDP-43 is a transactive response (TAR) DNA binding protein with Mw 43 kDa (TDP-43) and represents the major pathological marker, in both ALS and frontotemporal degeneration (FTD), in particular the most common pathological subtype of FTD, i.e., frontotemporal lobar degeneration with ubiquitinated inclusions, FTLD-U. Several missense mutations have been identified in TDP-43, the majority of which lie within the C-terminal region, indicating that this domain may be involved in the pathogenesis of ALS. Notably, TDP-43 pathology is associated with 97% of ALS (familial and sporadic) and 45% of frontotemporal dementia (FTD) cases.

Despite advances in understanding the physiology and pathophysiology of ALS and FTD, there is still a need for compounds that are potent, efficacious, and safe in the treatment or amelioration of ALS and FTD as well as other clinical conditions associated with a neurodegenerative disease or locomotor dysfunction or present TDP-43 pathology.

### SUMMARY OF THE INVENTION

Some aspects of this disclosure relate to treating a clinical condition associated with a neurodegenerative disease or locomotor dysfunction in a subject, said method comprising administering to the subject in need of such a treatment a therapeutically effective amount of a compound of the formula:

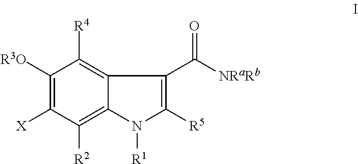

or a pharmaceutically acceptable salt thereof,
wherein
R¹ is hydrogen, alkyl, optionally substituted aralkyl, optionally substituted aryl, or a nitrogen protecting group;
each of R² and R⁴ is independently hydrogen, alkyl, haloalkyl, halide, vinyl, alkynyl, —CHO, —C(═O)R^{c} (ketone), —CO2R^{d} (ester), —OR^{e}, —OSO₂R^{f}, aryl and heteroaryl, wherein each of R^{c}, R^{d}, R^{e}, and R^{f} is independently alkyl or aryl;
each of R³ and R⁵ is independently alkyl or haloalkyl;
each of R^{a} and R^{b} is independently hydrogen or alkyl; and
X is halide, CN, OR^{x1}, NR^{x2}R^{x3} wherein each of R^{x1}, R^{x2}, R^{x3} are independently hydrogen, alkyl, cycloalkyl, optionally substituted aralkyl, optionally substituted aryl.

In some embodiments, R¹ is optionally substituted aralkyl. In one particular embodiment, R¹ is optionally substituted benzyl.

Still in other embodiments, at least one of R² and R⁴ is hydrogen. In further embodiments, both R² and R⁴ are hydrogen.

Yet in other embodiments, R³ is C₁-C₆ alkyl. In some instances, R³ is methyl or ethyl. In one particular embodiment, R³ is methyl.

In further embodiments, X is halide. Still in further embodiments, X is F or Cl. In one particular embodiment, X is F.

In other embodiments, at least one of R^{a} or R^{b} is hydrogen. Still in other embodiments, one of R^{a} and R^{b} is hydrogen and the other is an alky. Yet in further embodiments, R^{b} is alkyl, typically C₁-C₈ alkyl, often C₁-C₆ alkyl, and most often C₁-C₄ alkyl. In one particular embodiment, R^{b} is propyl.

Still in further embodiments, the neurodegenerative disease is selected from the group consisting of Alzheimer's disease, frontotemporal dementia, a frontotemporal dementia caused by mutations in progranulin protein, amyotrophic lateral sclerosis (ALS), Huntington's chorea, Creutzfeld-Jacob disease, trinucleotide repeat diseases, cerebral degenerative diseases presenile dementia, senile dementia, Parkinsonism linked to chromosome 17 (FTDP-17), progressive supranuclear palsy (PSP), Huntington's disease (HD), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarct, cerebral trauma, chronic traumatic encephalopathy, transient ischemic attack, encephalopathy, traumatic brain injury (TBI), and any combination thereof.

Yet in other embodiments, said clinical condition comprises Amyotrophic Lateral Sclerosis (ALS), frontotemporal degeneration (FTD), Alzheimer's Disease, encephalopathy, or traumatic brain injury (TBI). In one particular embodiment, said FTD comprises frontotemporal lobar degeneration with ubiquitinated inclusions. Still in other embodiments, said treatment comprises ameliorating a symptom associated with ALS.

Still in further embodiments, methods of the invention further comprise administering a compound that decreases release of glutamate to the subject. In one particular embodiment, said compound that decreases the release of glutamate comprises riluzole.

In further embodiments, methods of the invention include administering to said subject a compound comprising baclofen, trihexyphenidyl hydrochloride, morphine sulfate, lorazepam, glycopyrrolate, benztropine mesylate, gabapentin, diazepam, tizanidine, phenytoin sodium, amitriptyline hydrochloride, benztropine mesylate, ropinirole, glycopyrrolate, glycopyrrolate, Atropine sulphate, fluvoxamine maleate, dantrolene sodium, phenytoin sodium, gabapentin, morphine sulfate, dexpramipexole, or a combination of two or more compounds thereof.

Another aspect of the invention provides a compound of the formula:

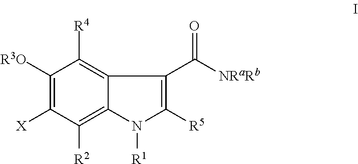

or a pharmaceutically acceptable salt thereof, or a combination thereof, thereby treating the clinical condition associated with locomotor dysfunction in said subject,
wherein
R¹ is hydrogen, alkyl, optionally substituted aralkyl, optionally substituted aryl, or a nitrogen protecting group;
each of R² and R⁴ is independently hydrogen, alkyl, haloalkyl, halide, vinyl, alkynyl, —CHO, —C(═O)R^{c} (ketone), —CO₂R^{d} (ester), —OR^{e}, —OSO₂R^{f}, optionally substituted aryl and optionally substituted heteroaryl, wherein each of R^{c}, R^{d}, R^{e}, and R^{f} is independently alkyl or aryl;
each of R³ and R⁵ is independently alkyl or haloalkyl;
each of R^{a} and R^{b} is independently hydrogen or alkyl; and
X is halide, CN, OR^{x1}, NR^{x2}R^{x3} wherein each of R^{x1}, R^{x2}, R^{x3} are independently hydrogen, alkyl, cycloalkyl, optionally substituted aralkyl, optionally substituted aryl.

In one particular embodiment, said compound is of the formula:

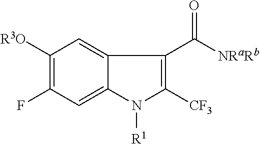

wherein R¹, R³, R^{a}, and R^{b} are those defined herein.

Still other aspects of the invention provide a composition comprising a compound of Formula I, and a pharmaceutically acceptable excipient.

### BRIEF DESCRIPTION OF THE DRAWINGS

FIG. 1 shows synthetic schemes for preparing various biotinylated analogs of one particular compound of the invention for studying structure activity relationship (SAR).

FIG. 2 is a graph showing results of locomotor function assay using Compound A.

FIGS. 3A-3F are bar graphs showing results of locomotor function assays using various biotinylated analogs of Compound H.

### DETAILED DESCRIPTION OF THE INVENTION

Long-standing observations made in the clinic point to defects in metabolic regulation including abnormalities in glucose and lipid metabolism in ALS. Using fruit fly model of ALS based on TDP-43, the present inventors have performed global metabolomics profiling and identified several significant metabolic changes consistent with alterations in cellular energetics. In particular, without being bound by any theory, it is believed that increased pyruvate in both TDPWT and disease-associated TDPG298S models is an indication of altered glucose metabolism. The present inventors have also found increased tricarboxylic (TCA) cycle intermediates are upregulated in plasma from ALS patients. Additionally, increased levels of fatty acid carnitine conjugates suggest decreased lipid beta-oxidation. Consistent with this observation, the ketone body marker 3-hydroxybutyrate (BHBA) is decreased, indicating impaired mitochondrial lipid metabolism and an increased reliance on glycolysis for energy production in affected motor neurons. Based at least in part on these results and findings by the present inventors that antidiabetic drugs show partial effectiveness in ALS, it is believed that improving glucose and lipid metabolism through diet and genetic intervention can provide protection against neurodegeneration.

The present inventors have also observed that various dietary and genetic interventions aimed at increasing ATP production mitigate TDP-43 dependent locomotor dysfunction and increase lifespan. Transcriptional profiling data for key glycolytic enzymes are consistent with increased glycolysis in both fly and patient-derived iPS motor neurons. Together with genetic interaction experiments, data collected to date support the notion that increased glycolysis is a compensatory mechanism, perhaps a last resort for motor neurons to counter the well documented reduction in cellular ATP/dysfunctional mitochondrial metabolism caused by TDP-43 toxicity. These findings indicate that glycolytic enzymes cluster at synapses and, under stress conditions, regulate synaptic vesicle endocytosis, a process that was found altered also through studies on hsc70-4 mRNA translation. To determine whether increased reliance on glycolysis is specific to TDP-43 or represents a common mechanism, a testing was conducted to determine whether a high glucose diet improves locomotor function and increases lifespan in additional models of ALS, based on SOD1 and C9ORF72. It was found that at least locomotor function was also improved in these other models by high glucose availability. Other experiments have been performed to determine the contribution of glucose and lipid metabolism to ALS phenotypes and to establish the feasibility of therapeutic strategies aimed at restoring energy homeostasis.

Compounds of the invention are derivatives or based at least in part on compounds disclosed in a commonly assigned U.S. patent application Ser. No. 16/515,743, filed Jul. 18, 2019, which is incorporated herein by reference, that showed neuroprotective activity in Drosophila models of ALS and patient derived motor neurons. In particular, compounds of the invention are of the formula:

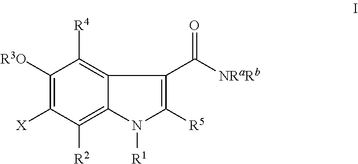

or a pharmaceutically acceptable salt thereof or a prodrug thereof, where X, R¹, R², R³, R⁴, R⁵, R^{a}, and R^{b} are those defined herein. In particular, compounds of the invention showed neuroprotective activity in Drosophila models of ALS and patient derived motor neurons. Accordingly, some aspects of the invention provide a method for treating a clinical condition associated with locomotor dysfunction in a subject by administering a therapeutically effective amount of compound of Formula I. With respect to compound of Formula I, in some embodiments, R¹ is hydrogen, alkyl, optionally substituted aralkyl, optionally substituted aryl, or a nitrogen protecting group. Still in other embodiments, each of R² and R⁴ is independently hydrogen, alkyl, haloalkyl, halide, vinyl, alkynyl, —CHO, —C(═O)R^{e}(ketone), —CO₂R^{d} (ester), —OR^{e}, —OSO₂R^{f}, aryl and heteroaryl, wherein each of R^{c}, R^{d}, R^{e}, and R^{f} is independently alkyl or aryl. In further embodiments, each of R³ and R⁵ is independently alkyl or haloalkyl. Yet in other embodiments, each of R^{a} and R^{b} is independently hydrogen or alkyl. Still yet in other embodiments, X is halide, typically F or Cl, and often X is F.

Administration of a therapeutically effective amount of a compound of Formula I afforded increased locomotor function. Accordingly, some aspects of the invention provide a method for treating a clinical condition associated with a neurodegenerative disease or locomotor dysfunction in a subject, said method comprising administering to the subject in need of such a treatment a therapeutically effective amount of a compound of Formula I.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and also includes any combination of members of that list.

When referring to a numerical value, the terms “about” and “approximately” are used interchangeably herein and refer to being within an acceptable error range for the particular value as determined by one of ordinary skill in the art, which will depend in part on how the value is measured or determined, e.g., the limitations of the measurement system, i.e., the degree of precision required for a particular purpose. In some embodiments, the term “about” typically means within 1 standard deviation, per the practice in the art. Alternatively, the term “about” can mean ±20%, typically ±10%, often ±5% and more often ±1% of the numerical value. In general, however, where particular values are described herein, unless otherwise stated, the term “about” means within an acceptable error range for the particular value.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g., an animal. In an aspect, the subject of the herein disclosed methods can be a Drosophila. In an aspect, the subject of the herein disclosed methods can be mammal, a fish, a bird, a reptile, or an amphibian. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. In an aspect, the subject is a mammal. A patient refers to a subject afflicted with a disease or disorder. The term “patient” includes human and veterinary subjects. In some aspects of the disclosed methods, the subject has been diagnosed with a need for treatment for amyotrophic lateral sclerosis, such as, for example, prior to the administering step.

As used herein, the term “treatment” refers to the medical management of a subject or a patient with the intent to cure, ameliorate, stabilize, mitigate, or prevent a disease, pathological condition, or disorder, such as, for example, ALS or FTD. This term includes active treatment, that is, treatment directed specifically toward the improvement, mitigation, and/or amelioration of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as ALS or FTD). In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving, ameliorating, or mitigating the disease, e.g., causing regression or further advancement of the disease. In an aspect, the disease, pathological condition, or disorder is amyotrophic lateral sclerosis.

As used herein, the term “prevent” or “preventing” refers to precluding, averting, obviating, forestalling, stopping, or hindering something from happening, especially by advance action. In an aspect, prevent or preventing refers to the ameliorating of one or more signs and symptoms associated with ALS or FTD. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by compositions or methods disclosed herein. For example, “diagnosed with amyotrophic lateral sclerosis” means having been subjected to a physical examination by a person of skill, for example, a physician, and found to have a condition that can be diagnosed or treated by a compound or composition that alleviates or ameliorates one or more symptoms associated with amyotrophic lateral sclerosis.

Some aspects of the invention provide a method for diagnosing for the presence of TDP-43 in a sample such that the expression level or expression pattern of TDP-43 indicates to a clinician that the sample is indicative of a clinical condition associated with a neurodegenerative disease or a locomotor dysfunction. Diagnosing can further include comparing the level or binding pattern of TDP-43 to a reference sample that indicates that the subject (or sample) suffers from (contains) a TDP-43 proteinopathy. TDP-43 aggregates are associated with TDP-43 proteinopathy. Diagnosing can also include identifying mutations (e.g., mutations in SOD1 or FUS) by sequencing that indicates to a clinician that the sample is indicative of a clinical condition associated with a neurodegenerative disease or a locomotor dysfunction such as ALS.

Some embodiments of such methods comprise determining the expression level of TDP-43 in a sample obtained from the subject. As used herein, the term “expression” refers to (1) detecting transcription and/or translation of TDP-43 gene, (2) detecting or determining the amount of TDP-43 present in the sample, or (3) both. To detect expression of a gene refers to the act of actively determining whether a gene is expressed or not. This can include determining whether the gene expression is upregulated as compared to a control, downregulated as compared to a control, or substantially unchanged as compared to a control. Therefore, the step of detecting expression does not require that expression of the gene actually is upregulated or downregulated, but rather, can also include detecting no expression of the gene or detecting that the expression of the gene has not changed or is not different (i.e., detecting no significant expression of the gene or no significant change in expression of the gene as compared to a control).

According to the invention, a “control” can include a normal or negative control and/or a disease or positive control, against which a test level of TDP-43 expression can be compared. Therefore, it can be determined, based on the control expression level of TDP-43, whether a sample to be evaluated for a clinical condition associated with a neurodegenerative disease or a locomotor dysfunction has a measurable difference or substantially no difference in the TDP-43 expression level, as compared to the control level. In one aspect, the control is an indicative of the expression level of TDP-43 as expected in a normal (e.g., healthy, negative control) patient.

As used herein, the phrase “identified to be in need of treatment for a disorder,” or the like, refers to selection of a subject based upon need for treatment of the disorder. For example, a subject can be identified as having a need for treatment of a neurodegenerative disorder (e.g., amyotrophic lateral sclerosis) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, intracardiac administration, oral administration, transdermal administration, administration by inhalation, nasal administration, topical administration, ophthalmic administration, intraaural administration, intracerebral administration, sublingual administration, buccal administration, and parenteral administration, including injectable such as intravenous administration, intra-arterial administration, intramuscular administration, and subcutaneous administration. In an aspect, administering can refer to oral administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition, such as, for example, amyotrophic lateral sclerosis.

The term “contacting” as used herein in reference to a treatment refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “determining” can refer to measuring or ascertaining a quantity or an amount or a change in expression and/or activity level, e.g., of a nucleotide or transcript or polypeptide. For example, determining the amount of a disclosed transcript or polypeptide in a sample as used herein can refer to the steps that the skilled person would take to measure or ascertain some quantifiable value of the transcript or polypeptide (e.g., TDP-43) in the sample. The art is familiar with the ways to measure an amount of the disclosed nucleic acids, transcripts, polypeptides, etc.

The term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample).

The terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

The term “pharmaceutically acceptable” describes a material that is not biologically or otherwise undesirable, i.e., without causing an unacceptable level of undesirable biological effects or interacting in a deleterious manner. The term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

“Alkyl” refers to a saturated linear monovalent hydrocarbon moiety of one to twelve, typically one to six, carbon atoms or a saturated branched monovalent hydrocarbon moiety of three to twelve, preferably three to six, carbon atoms. Exemplary alkyl group include, but are not limited to, methyl, ethyl, n-propyl, 2-propyl, tert-butyl, pentyl, and the like.

“Alkylene” refers to a saturated linear divalent hydrocarbon moiety of one to twelve, typically one to six, carbon atoms or a branched saturated divalent hydrocarbon moiety of three to twelve, preferably three to six, carbon atoms. Exemplary alkylene groups include, but are not limited to, methylene, ethylene, propylene, butylene, pentylene, and the like.

“Alkynyl” refers to a hydrocarbon moiety having at least one carbon-carbon triple bond. Typically, the number of carbon atoms is from two to twenty, often two to twelve, more often two to eight, and most often two to six.

“Vinyl” refers to a moiety —C═CH.

“Aryl” refers to a monovalent mono-, bi- or tricyclic aromatic hydrocarbon moiety of 6 to 15 ring atoms. “Optionally substituted aryl” refers to an aryl group that is optionally substituted with one or more, preferably one, two, or three substituents within the ring structure. When two or more substituents are present in an aryl group, each substituent is independently selected. Exemplary substituents for the aryl group include, but are not limited to, alkyl, haloalkyl, thioalkyl, heteroalkyl, halo, nitro, cyano, cycloalkyl, aryl, heteroaryl, heterocyclyl, haloalkoxy, aryloxy, heteroaryloxy, etc.

“Aralkyl” refers to a moiety of the formula —R^{b}R^{c} where R^{b} is an alkylene group and R^{c} is an aryl group as defined herein. Exemplary aralkyl groups include, but are not limited to, benzyl, phenylethyl, 3-(3-chlorophenyl)-2-methylpentyl, and the like. The term “optionally substituted aralkyl” means the aryl group is optionally substituted with one or more, typically, one to three, and often one or two, substituents. Exemplary substituents for the aryl group include, but are not limited to, alkyl, haloalkyl, thioalkyl, heteroalkyl, halo, nitro, cyano, cycloalkyl, aryl, heteroaryl, heterocyclyl, haloalkoxy, aryloxy, heteroaryloxy, etc.

The terms “cycloalkyl” and “cyclyl” are used interchangeably herein and refer to a saturated mono-, bi-, or tri-cyclic monovalent hydrocarbon moiety. Unless the number of carbon atoms is specified, the term “cycloalkyl” refers to hydrocarbon moiety having a ring structure of from three to twenty, typically three to twelve, often three to eight, and more often three to six carbon atoms. Exemplary cycloalkyl groups include, but are not limited to, cyclopropyl, cyclobutyl, cyclopentyl, cyclohexyl, cyclooctyl, cyclododecyl, adamantanyl, norbornyl, and the like.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

The term “heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring is optionally substituted independently with one or more substituents, preferably one or two substituents, selected from alkyl, haloalkyl, heteroalkyl, heterocyclyl, halo, nitro, cyano, carboxy, acyl, -(alkylene)ₙ-COOR (where n is 0 or 1 and R is hydrogen, alkyl, optionally substituted phenylalkyl, or optionally substituted heteroaralkyl), or -(alkylene)ₙ-CONR^{a}R^{b} (where n is 0 or 1, and R^{a} and R^{b} are, independently of each other, hydrogen, alkyl, cycloalkyl, cycloalkylalkyl, hydroxyalkyl, aryl, or R^{a} and R^{b} together with the nitrogen atom to which they are attached form a heterocyclyl ring). More specifically the term heteroaryl includes, but is not limited to, pyridyl, furanyl, thiophenyl, thiazolyl, isothiazolyl, triazolyl, imidazolyl, oxazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, thiazolyl, isothiazolyl, quinolyl, isoquinolyl, benzimidazolyl, benzisoxazolyl, benzothiophenyl, dibenzofuran, and benzodiazepin-2-one-5-yl, and the like.

“Protecting group” refers to a moiety, except alkyl groups, that when attached to a reactive group in a molecule mask, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P or S) to which it is attached.

When describing a chemical reaction, the terms “treating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

As used herein, the terms “those defined above,” “those defined herein,” “as defined above”, and “as defined herein” are used interchangeably and when referring to a variable incorporates by reference the broad definition of the variable as well as any narrow and/or preferred, more preferred and most preferred definitions, if any.

Compounds of the Invention: Some aspects of the invention provide compound of Formula I or a pharmaceutically acceptable salt thereof, or a prodrug thereof. Compounds of the invention can be readily prepared using methods disclosed in the commonly assigned U.S. patent application Ser. No. 16/515,743, which is incorporated by reference in its entirety.

Salts are physiologically acceptable salts of the compounds disclosed herein. Physiologically acceptable salts of the compounds disclosed herein include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

The terms “pro-drug” and “prodrug” are used interchangeably herein and refer to any compound which releases an active parent drug according to Formula I in vivo when such prodrug is administered to a mammalian subject. Prodrugs of a compound of Formula I are prepared, for example, by modifying one or more functional group(s) present in the compound of Formula I in such a way that the modification(s) may be cleaved in vivo to release the parent compound. Prodrugs include compounds of Formula I wherein a hydroxy, amino, or sulfhydryl group in a compound of Formula I is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of Formula I, and the like.

Administration and Pharmaceutical Composition: The compounds described herein can be used in mitigating locomotor dysfunction, reducing TDP-43 aggregation or toxicity, ameliorating one or more symptoms associated with ALS, or to decrease the release of glutamate.

Compounds and compositions disclosed herein can also be used for the treatment of one or more neurodegenerative diseases. The compounds disclosed herein can be used to prevent a neurodegenerative disease, ameliorating one or more symptoms associated with one or more neurodegenerative diseases, or to decrease the release of glutamate.

Accordingly, another aspect of the invention includes pharmaceutical compositions for treating a various clinical conditions associated with a neurodegenerative disease or locomotor dysfunction in a subject. The pharmaceutical compositions disclosed herein comprise at least one compound of the invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention are administered in a therapeutically effective amount by any of the accepted modes of administration for agents that serve similar utilities. Suitable dosage ranges are typically 1-500 mg daily, typically 1-100 mg daily, and often 1-30 mg daily, depending on numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases is typically able, without undue experimentation and in reliance upon personal knowledge and the disclosure of this application, to ascertain a therapeutically effective amount of the compounds of the invention.

Typically, compounds of the invention are administered as pharmaceutical formulations including those suitable for oral (including buccal and sub-lingual) or parenteral (including intramuscular, intraarterial, intrathecal, subcutaneous and intravenous) administration or in a form suitable for administration by inhalation or insufflation. Typical manner of administration is generally oral or parenteral using a convenient daily dosage regimen which can be adjusted according to the degree of affliction.

A compound or compounds of the invention, together with one or more conventional adjuvants, carriers, or diluents, can be placed into the form of pharmaceutical compositions and unit dosages. The pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention can be formulated in a wide variety of oral administration dosage forms. The pharmaceutical compositions and dosage forms can comprise a compound or compounds of the invention or pharmaceutically acceptable salts thereof as the active component. The pharmaceutically acceptable carriers can be either solid or liquid. Solid form preparations include powders, tablets, pills, capsules, cachets, suppositories, and dispersible granules. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

The compounds of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water.

The compounds of the invention can be formulated for topical administration to the epidermis as ointments, creams or lotions, or as a transdermal patch. Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention can be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

The compounds of the invention can be formulated for aerosol administration, particularly to the respiratory tract and including intranasal administration. The compound will generally have a small particle size for example of the order of five (5) microns or less. Such a particle size can be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol can conveniently also contain a surfactant such as lecithin. The dose of drug can be controlled by a metered valve. Alternatively, the active ingredients can be provided in a form of a dry powder, for example, a powder mix of the compound in a suitable powder base such as lactose, starch, starch derivatives such as hydroxypropylmethyl cellulose and polyvinylpyrrolidine (PVP). The powder carrier typically forms a gel in the nasal cavity. The powder composition can be presented in unit dose form, for example, in capsules or cartridges of e.g., gelatine or blister packs from which the powder can be administered by means of an inhaler.

When desired, formulations can be prepared with enteric coatings adapted for sustained or controlled release administration of the active ingredient. For example, the compounds of the invention can be formulated in transdermal or subcutaneous drug delivery devices. These delivery systems are advantageous when sustained release of the compound is necessary or desired and when patient compliance with a treatment regimen is crucial. Compounds in transdermal delivery systems are frequently attached to a skin-adhesive solid support. The compound of interest can also be combined with a penetration enhancer, e.g., Azone (1-dodecylazacycloheptan-2-one). Sustained release delivery systems can be inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are typically in unit dosage forms. In such form, the preparation is often subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

When it is possible that, for use in therapy, therapeutically effective amounts of a compound of Formula (I), as well as pharmaceutically acceptable salts thereof, can be administered as the raw chemical, it is possible to present the active ingredient as a pharmaceutical composition. Accordingly, the disclosure further provides pharmaceutical compositions, which include therapeutically effective mounts of compounds of Formula (I) or pharmaceutically acceptable salts thereof or a prodrug thereof, and one or more pharmaceutically acceptable carriers, diluents, or excipients. When applied to a combination, the term refers to combined amounts of the active ingredients that result in the therapeutic effect, whether administered in combination, serially, or simultaneously. The carrier(s), diluent(s), or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound disclosed herein, or a pharmaceutically acceptable salt thereof or a prodrug thereof, with one or more pharmaceutically acceptable carriers, diluents, or excipients.

When the compositions of this disclosure comprise a combination of a compound of the present disclosure and one or more additional therapeutic or prophylactic agent, both the compound and the additional agent are usually present at dosage levels of between about 10 to 150%, and more typically between about 10 and 80% of the dosage normally administered in a monotherapy regimen.

Utility: Compositions and compounds of the invention are useful in treating a variety of clinical conditions such as, but not limited to, a neurodegenerative disease and locomotor dysfunction. Exemplary neurodegenerative diseases that can be treated using a composition and/or a compound of the invention include, but are not limited to, Alzheimer's disease, frontotemporal dementia, FTLD-U (a frontotemporal dementia caused by mutations in progranulin protein), amyotrophic lateral sclerosis (ALS), Huntington's chorea, Creutzfeld-Jacob disease, trinucleotide repeat diseases, cerebral degenerative diseases presenile dementia, senile dementia, Parkinsonism linked to chromosome 17 (FTDP-17), progressive supranuclear palsy (PSP), Huntington's disease (HD), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarct, cerebral trauma, chronic traumatic encephalopathy, transient ischemic attack, encephalopathy, and traumatic brain injury (TBI).

Amyotrophic Lateral Sclerosis (ALS): ALS is a rapidly progressing neurodegenerative disease with currently no curative treatment. Using a previously developed a Drosophila (fruit fly) model of ALS, compounds of the invention were identified that can reduce the locomotor dysfunction associated with the disease. Results show that these compounds mitigate TDP-43 proteinopathy which is present in 97% of ALS patients, 45% of FTD patients and a growing number of patients with related neurodegenerative disorders including Alzheimer's PD, etc. TDP-43 proteinopthy manifests in part as mislocalization to the cytoplasm (sometimes accompanied by aggregation) is one of the characteristics of ALS and FTD.

Amyotrophic lateral sclerosis (ALS) is a rapidly progressive, invariably fatal neurological disease that attacks the neurons responsible for controlling voluntary muscles, such as those in the arms, legs and face. ALS causes weakness with a wide range of disabilities. Eventually, all muscles under voluntary control are affected, and individuals lose their strength and the ability to move their arms, legs, and body. When muscles in the diaphragm and chest wall fail, people lose the ability to breathe without ventilatory support. Most people with ALS die from respiratory failure, usually within 3 to 5 years from the onset of symptoms. However, about 10 percent of those with ALS survive for 10 or more years.

Currently no cure that can reverse the damage of ALS exists. The drugs riluzole (Rilutek®, Sanofi) and Radicava™ (edaravone) are the only medications approved by the FDA for ALS. Without being bound by any theory, it is believed that riluzole reduces damage to motor neurons by decreasing glutamate toxicity at synapses while edaravone is believed to act as a free radical scavenger although its mechanism of action in ALS remains unknown. Neither Riluzole nor Radicava, however, can reverse the damage already done to motor neurons, and persons taking the drug must be monitored for liver damage and other possible side effects. ALS has a high level of unmet need, given that an optimal course of therapy can extend a patient's survival by only a few months.

Other marketed products used as treatments for ALS include baclofen, trihexyphenidyl hydrochloride, morphine sulfate, lorazepam, glycopyrrolate, benztropine mesylate, gabapentin, Valium (diazepam), tizanidine, phenytoin sodium, Elavil (amitriptyline hydrochloride), Cogentin (benztropine mesylate), ReQuip, Robinul (glycopyrrolate), Cuvposa (glycopyrrolate), Atropine sulphate, Luvox (fluvoxamine maleate), Dantrium (dantrolene sodium), Dilantin (phenytoin sodium), Neurontin (gabapentin), morphine sulfate and dexpramipexole.

While multiple drugs have shown promise in preclinical in vitro and in vivo models of ALS, they have failed to show efficacy in human trials. These include glutamate antagonists other than riluzole, neurotrophic factors, antiapoptotic agents, antioxidants, and immunomodulatory drugs.

TDP-43: TDP-43 is a conserved RNA binding protein involved in several cellular processes. TDP-43 has been initially identified as a nucleic acid binding protein that regulates HIV gene expression by binding to its TAR DNA element, hence its original name, TAR DNA Binding Protein (TARDP). TDP-43 is ubiquitously expressed and co-localizes with SMN proteins in the nucleus. Its cellular functions include transcriptional repression, splicing, miRNA biogenesis, apoptosis and cell division. TDP-43 associates with RNA granules and co-purifies with β-actin and CaMKII mRNAs in cultured neurons. TDP-43 co-localizes with Fragile X protein and Staufen in an activity dependent manner, indicating that it can regulate synaptic plasticity in vivo by controlling the transport, splicing and translation of synaptic mRNAs. TDP-43 protein consists of two RNA recognition motifs (RRM1 and 2) as well as a Glycine-rich domain within the C terminus. In vitro assays have demonstrated that TDP-43 binds with high affinity UG-rich sequences, consistent with its role in mRNA splicing.

Pathological studies have identified the RNA binding protein TDP-43 as a component of cytoplasmic aggregates in neurons, glia and muscles, in ALS as well as Fronto-Temporal Lobar Degeneration, Alzheimer's and Inclusion Body Myositis. TDP-43's cellular functions are complex and reflect its ability to regulate several RNA targets at the level of splicing, transport and translation. Recently, several TDP-43 mutations have been identified in ALS patients. Given its presence in cytoplasmic inclusions and the identification of several mutations linked to motor neuron degeneration, TDP-43 has emerged as a common denominator for a significant fraction of ALS cases known to date. TDP-43's ability to form aggregates, induce apoptosis and splicing function can be used as secondary assays.

Most TDP-43 mutations found in ALS patients represent amino acid substitutions that are thought to increase TDP-43 phosphorylation and target it for degradation. Evidence has been provided that these missense mutations mimic a loss of nuclear function and a gain of cytoplasmic function for TDP-43, and that the RNA binding domain is required to mediate neurotoxicity. Given its presence in cytoplasmic inclusions and the identification of several mutations linked to neural degeneration, TDP-43 has emerged as a common denominator for the majority of ALS cases known to date. Thus, studies using this model of ALS based on TDP-43 can provide widely applicable insights into this disease.

In recent years, the fruit fly Drosophila has emerged as a premiere genetic model for studying human disease. Using loss of function mutations in Drosophila TDP-43 (dTDP-43) as well as RNAi knock-down approaches, alterations were identified in the architecture of the larval neuromuscular junction, locomotor defects and retina neurodegeneration in adults. To further establish a Drosophila model, transgenics were generated expressing wild-type and mutant forms of fly and human TDP-43, which mimic the mutations found in human patients. Experiments show that overexpression of TDP-43 results in the formation of cytoplasmic aggregates, neuronal loss and locomotor defects. Taken together, these data indicate that loss of function for TDP-43 as well as overexpression of wild-type and mutant TDP-43 in Drosophila recapitulate several aspects of the disease pathology. Furthermore, immunolocalization and genetic rescue data indicate a loss of function mechanism for the disease. The defects due to altered TDP-43 function can be corrected by genetic and pharmacological intervention and the genes/compounds that rescue the TDP-43 phenotypes can provide new therapeutic approaches for ALS. Genetic screens have identified a number of candidate genomic regions that rescue the retinal degeneration due to TDP-43 misexpression in the eye and/or locomotor dysfunction caused by TDP-43 proteinopathy.

Since TDP-43 is involved in the pathology of a majority of ALS cases and has recently been shown to also be a cause for the disease, studies of a TDP-43 based model can provide insights into a wide spectrum of ALS cases. There is data indicating that in flies, alterations in TDP-43 function lead to defects in neuromuscular junction architecture, adult locomotion and neurodegeneration. Several transgenic lines expressing wild-type and mutant forms of dTDP-43 and human TDP-43 (huTDP-43) that correspond to mutations found in human patients have been developed. Overexpression of these transgenes leads to the formation of cytoplasmic inclusions and leads to neuronal dysfunction and death. The data demonstrates that the fly model recapitulates several aspects of ALS pathology. Genetic screens can identify single gene mutations that can rescue or enhance the TDP-43 phenotypes. These genes can provide insights into the molecular mechanisms involved in ALS and may represent novel therapeutic targets. In addition, this approach has the potential to identify ALS loci that are yet to be discovered in human patients. TDP-43-based phenotypes can be used to screen for compounds that rescue the neuroanatomical and functional defects in the fly model. It should be noted that this fly model has been validated in human samples.

It has been shown that ALS can be successfully modeled in Drosophila using both loss of function and gain of function approaches. Transgenic lines, wild-type and several mutant forms of both the fly and human proteins, which can be expressed specifically in motor neurons and the eye neuroepithelium using the Gal4-UAS system have been produced. Phenotypic analyses including testing for cytoplasmic inclusions, neuroanatomical and functional studies can determine the extent to which different alterations in TDP-43 recapitulate the pathology associated with the human disease.

ALS is a group of complex motor neuron diseases involving a dozen distinct and overlapping protein inclusions, including TAR DNA-binding protein-43 (TDP-43). TDP-43 is found in 97% of patients with ALS. Described herein are results using a recently developed Drosophila (fruit fly) model for ALS, by expressing human transactive response DNA-binding protein 43 kDA (TAR DNA-binding protein 43, TDP-43) to test the compounds disclosed herein. TDP-43 is involved in RNA processing, including splicing, transcription, translation and transport. The primary histopathological feature in a major subset of ALS cases is the inclusion of TDP-43 in the cytoplasm of upper and lower motor neurons and in other regions of the central nervous system. While TDP-43 aggregation (or toxicity) is a hallmark of most of the ALS cases, it is also found in neurodegenerative disorders, including frontotemporal dementia and Alzheimer's disease.

Protein aggregation is a pathology characteristic of ALS. SOD1 was the first protein to be identified to aggregate in familial ALS cases carrying a mutation in SOD1 gene. Due to exponential development of genetic techniques, several new proteins have been identified to be involved in ALS pathophysiology during the past few years, including TDP-43, FUS, OPTN, UBQLN2 and C9ORF72.

In some aspects of the invention, compounds and compositions disclosed herein can be used to target TDP-43 aggregation or toxicity for treatment of ALS and neurodegenerative diseases. In some embodiments, the compounds and compositions described herein target TDP-43 and may be useful neuroprotective agents for TDP-43 proteinopathies.

As stated herein, the compounds disclosed herein can be used to treat one or more neurodegenerative diseases, including but not limited to Alzheimer's disease, frontotemporal dementia, FTLD-U (a frontotemporal dementia caused by mutations in progranulin protein), amyotrophic lateral sclerosis (ALS), Huntington's chorea, Creutzfeld-Jacob disease, trinucleotide repeat diseases, cerebral degenerative diseases presenile dementia, senile dementia, Parkinsonism linked to chromosome 17 (FTDP-17), progressive supranuclear palsy (PSP), Huntington's disease (HD), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarct, cerebral trauma, chronic traumatic encephalopathy, and transient ischemic attack.

In an aspect, the compounds or compositions described herein further comprise a detectable label. Detectable labels include, but are not limited to, any detectable moiety, including, for example, fluorescent labels, biotinylated labels, radioactive labels, and electronic labels. In an aspect, the labels can be used to detect or identify TDP-43 in the plasma, cerebrospinal fluid, brain, spinal cord or other samples. In an aspect, detecting and monitoring levels or aggregation (or toxicity) of TDP-43 in ALS or other neurodegenerative diseases can provide the ability to diagnose and distinguish TDP-43 proteinopathies from other clinically similar neurodegenerative diseases (e.g., tauopathies or other proteinopathies). Further, detection of and/or quantification of TDP-43 neuropathology in living patients can also provide a diagnosis and the ability to monitor the response of patients having a neurodegenerative TDP-43 proteinopathy to disease-modifying therapies. In an aspect, the detectable label can be used to detect the presence of TDP-43 in a sample in which a compound specifically binds TDP-43.

Disclosed herein are methods of using one or more the compounds disclosed herein for the treatment or prevention of diseases or disorders, including one or more of the diseases or disorders described above.

Also disclosed herein are methods of mitigating locomotor dysfunction in a subject in need thereof. The methods include the step of administering to the subject a therapeutically effective amount of at least one compound of Formula I or a pharmaceutically acceptable salt thereof or a prodrug thereof. In some embodiments, the method utilizes at least one compound of Formula I, or a pharmaceutically acceptable salt thereof, or a prodrug thereof.

Other aspects of the invention include reducing TDP-43 aggregation or toxicity in a subject in need thereof. The methods include the step of administering to the subject a therapeutically effective amount of at least one compound of Formula I, or a pharmaceutically acceptable salt thereof, or a prodrug thereof, thereby reducing TDP-43 aggregation or toxicity in the subject. In some embodiments, the TDP-43 expression itself is not reduced, i.e., as compared to the TDP-43 expression level prior to administration of a compound of the invention. It should be appreciated that the term “not reduced” means there is no statistically significant difference in TDP-43 expression level. In some embodiments, the method utilizes at least one compound of Formula I, or a pharmaceutically acceptable salt thereof, or a prodrug thereof.

Another aspect of the invention provides a method for treating ALS in a subject in need thereof. The methods include the step of administering to the subject a therapeutically effective amount of at least one compound of Formula I, or a pharmaceutically acceptable salt thereof, or a prodrug thereof, thereby treating ALS in the subject.

Yet another aspect of the invention provides methods for ameliorating a symptom associated with ALS in a subject in need thereof. The methods include the step of administering to the subject a therapeutically effective amount of at least one compound of Formula I, or a pharmaceutically acceptable salt thereof, or a prodrug thereof, thereby ameliorating a symptom associated with ALS in the subject.

Still yet another aspect of the invention provides a method for treating a neurodegenerative disease in a subject in need thereof. The methods include the step of administering to the subject a therapeutically effective amount of at least one compound of Formula I, or a pharmaceutically acceptable salt thereof, or a prodrug thereof, thereby treating a neurodegenerative disease in the subject. In some embodiments, the neurodegenerative disease is selected from the group consisting of Alzheimer's disease, frontotemporal dementia, FTLD-U (a frontotemporal dementia caused by mutations in progranulin protein), amyotrophic lateral sclerosis (ALS), Huntington's chorea, Creutzfeld-Jacob disease, trinucleotide repeat diseases, cerebral degenerative diseases presenile dementia, senile dementia, Parkinsonism linked to chromosome 17 (FTDP-17), progressive supranuclear palsy (PSP), Huntington's disease (HD), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarct, cerebral trauma, chronic traumatic encephalopathy, and transient ischemic attack, or any combination thereof.

Another aspect of the invention provides methods for preventing a neurodegenerative disease in a subject in need thereof. The methods include the step of administering to the subject a therapeutically effective amount of at least one compound of Formula I, or a pharmaceutically acceptable salt thereof, or a prodrug thereof, thereby preventing a neurodegenerative disease in the subject.

In yet another aspect of the invention, in any of the methods disclosed herein, the methods can further comprise administering a therapy that relieves one or more symptoms associated with a clinical condition to be treated. As an example, symptoms of ALS include but are not limited to stiff muscles, muscle twitching, muscle weakness due to muscles decreasing in size, difficulty speaking, swallowing and breathing. Thus, a method for treating ALS disclosed herein can also include a combination therapy in which a therapy (or a compound) that decreases the release of glutamate can be co-administered either simultaneously or separately. In one particular embodiment, in any of the methods disclosed herein, the methods can further comprise administering a compound that decreases the release of glutamate to the subject. In another embodiment of the invention, in any of the methods disclosed herein, the methods can further comprise administering to the subject one or more of baclofen, trihexyphenidyl hydrochloride, morphine sulfate, lorazepam, glycopyrrolate, benztropine mesylate, gabapentin, Valium (diazepam), tizanidine, phenytoin sodium, Elavil (amitriptyline hydrochloride), Cogentin (benztropine mesylate), ReQuip, Robinul (glycopyrrolate), Cuvposa (glycopyrrolate), Atropine sulphate, Luvox (fluvoxamine maleate), Dantrium (dantrolene sodium), Dilantin (phenytoin sodium), Neurontin (gabapentin), morphine sulfate and dexpramipexole.

In some embodiments, the subject is a mammal. In one particular embodiment, the mammal is a human.

The compounds disclosed herein can be used alone or, if required, in combination with other active compounds. Accordingly, disclosed herein are medicaments comprising at least one of the compounds described herein and one or more further active compounds for the treatment and/or prevention of one or more of the diseases or disorders mentioned above. Suitable active compounds for combinations are one or more of baclofen, trihexyphenidyl hydrochloride, morphine sulfate, lorazepam, glycopyrrolate, benztropine mesylate, gabapentin, Valium (diazepam), tizanidine, phenytoin sodium, Elavil (amitriptyline hydrochloride), Cogentin (benztropine mesylate), ReQuip, Robinul (glycopyrrolate), Cuvposa (glycopyrrolate), Atropine sulphate, Luvox (fluvoxamine maleate), Dantrium (dantrolene sodium), Dilantin (phenytoin sodium), Neurontin (gabapentin), morphine sulfate and dexpramipexole.

Disclosed herein are medicaments comprising at least one compound described herein, usually in combination with one or more inert, non-toxic, pharmaceutically suitable excipients, and their use for the purposes mentioned above. The compounds according to the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or optic route or as implant or stent.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### General Procedure for Fluorinated Indoles

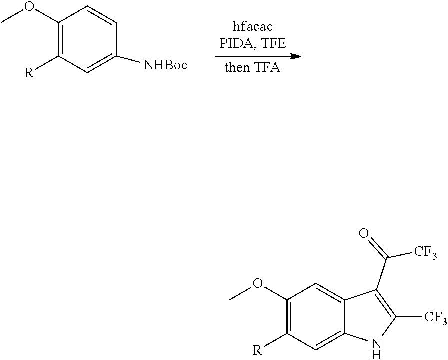

To a 20 mL vial equipped with a stir bar, N-Boc p-anisidine derivative (1 equiv) and a septum, 2,2,2-trifluoroethanol (TFE) (0.25M overall concentration) was added and the solution purged and maintained under nitrogen. To this solution, hexafluoroacetylacetone (hfacac) (10 equiv) was syringed in, followed by the addition of phenyliodine(III) diacetate (PIDA) (2 equiv) in TFE via a syringe over 30 seconds, and the reaction was stirred for 30 minutes. Trifluoroacetic acid (TFA) (10 equiv) was syringed in, the septum was replaced with a Teflon-lined cap, and the reaction was stirred overnight (10-12 hours). The reaction mixture was then concentrated in vacuo, and the residue was purified by silica gel flash column chromatography (3:15:85 AcOH/EtOAc/hexanes).

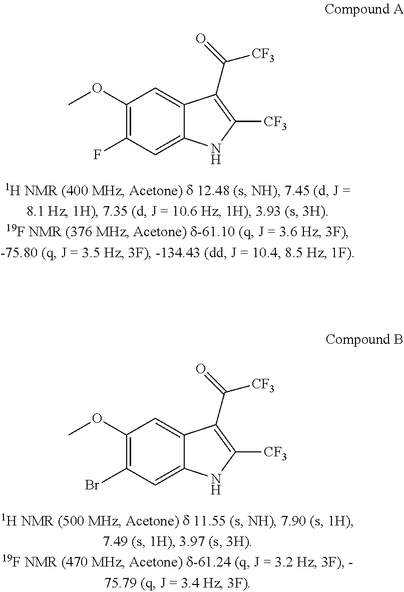 [SMILES: COc1cc2c(C(=O)C(F)(F)F)c(C(F)(F)F)[nH]c2cc1F ; COc1cc2c(C(=O)C(F)(F)F)c(C(F)(F)F)[nH]c2cc1Br]

General Procedure for N-Alkylation:

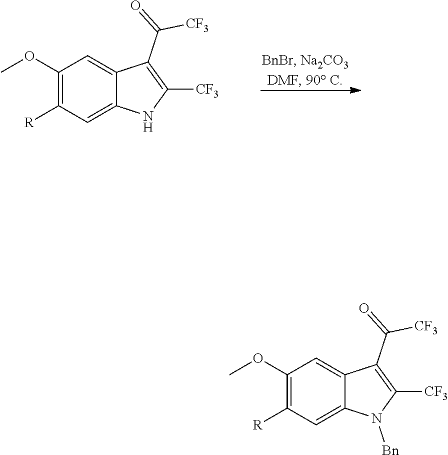

To a flame dried 50 mL round-bottom as equipped with a stir bar an septum, fluorinated indole derivative (1 equiv), sodium carbonate (2 equiv) and dry DMF (0.25 M) were added and the reaction purged and maintained under nitrogen. The reaction mixture was stirred 5 min before adding benzyl bromide (2 equiv) all at once. The reaction was stirred 1 hour at room temperature, then placed in an oil bath preheated to 90° C. and stirred overnight. The reaction was cooled to room temperature and brine was added, and the product was extracted with EtOAc. The organic fractions were combined, dried with anhydrous Na₂SO₄, filtered, and concentrated in vacuo. The crude product was purified by silica gel flash column chromatography (2:15:85 Et₃N/EtOAc/hexanes).

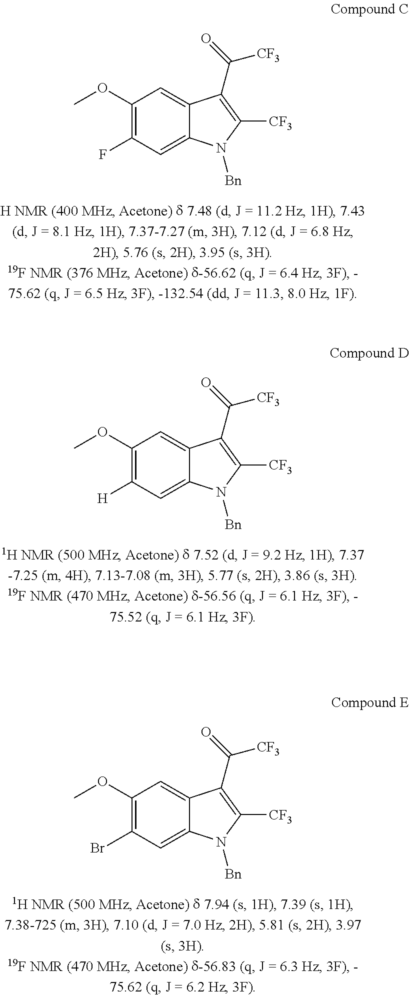 [SMILES: COc1cc2c(C(=O)C(F)(F)F)c(C(F)(F)F)n(Cc3ccccc3)c2cc1F ; COc1ccc2c(c1)c(C(=O)C(F)(F)F)c(C(F)(F)F)n2Cc1ccccc1 ; COc1cc2c(C(=O)C(F)(F)F)c(C(F)(F)F)n(Cc3ccccc3)c2cc1Br]

General Procedure for Hydrolysis

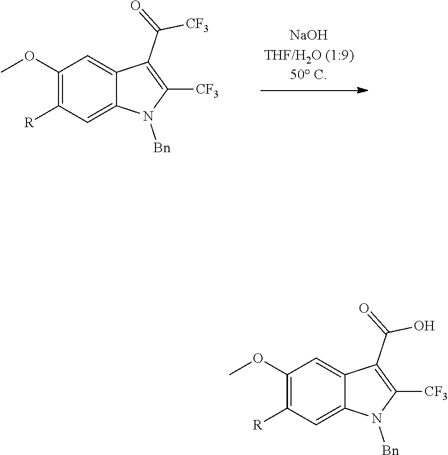

To a 20 mL vial equipped with a stir bar, N-Bn indole derivative (1 equiv), sodium hydroxide (10 equiv) and THF/H2O (1:9, 0.323M) were added and the reaction was sealed and stirred at 50° C. overnight. The reaction was cooled to room temperature, acidified with 6 N aqueous HCl and extracted with EtOAc. The organic fractions were combined, dried with anhydrous Na₂SO₄, filtered, and concentrated in vacuo. The crude solid was rinsed with chloroform to give the product

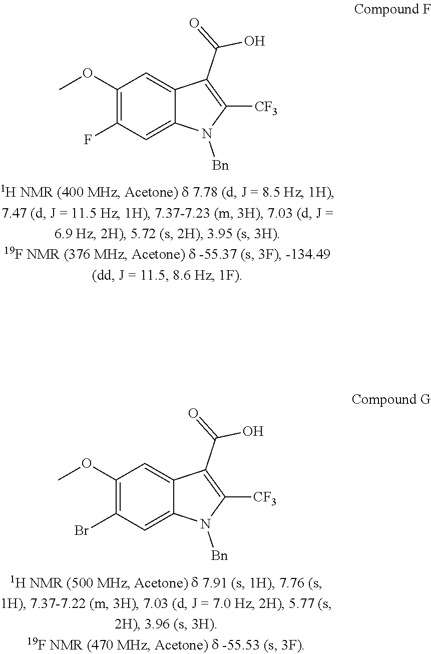 [SMILES: COc1cc2c(C(=O)O)c(C(F)(F)F)n(Cc3ccccc3)c2cc1F ; COc1cc2c(C(=O)O)c(C(F)(F)F)n(Cc3ccccc3)c2cc1Br]

General Procedure for Amide Formation

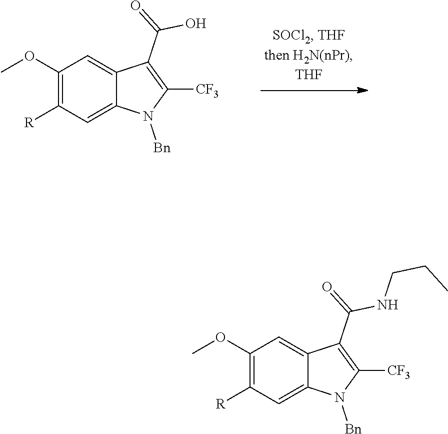

To a flame dried 4 mL vial equipped with a stir bar and septum, indole-3-carboxylic acid derivative (1 equiv) and THE (0.1M) were added and the solution purged and maintained under nitrogen. To this solution was added thionyl chloride (12.5 equiv) and the mixture was stirred at room temperature for 1 h, and concentrated in vacuo. The residue was dissolved in THE (0.075M) and the solution was purged and maintained under nitrogen. To this solution was added propylamine (26.7 equiv) at room temperature. After stirring at room temperature for 1 h, brine was added and the product extracted with EtOAc. The combined organic layers were dried with anhydrous Na₂SO₄, filtered, and concentrated in vacuo. The crude product was purified by silica gel flash column chromatography (15-30% EtOAc/hexanes).

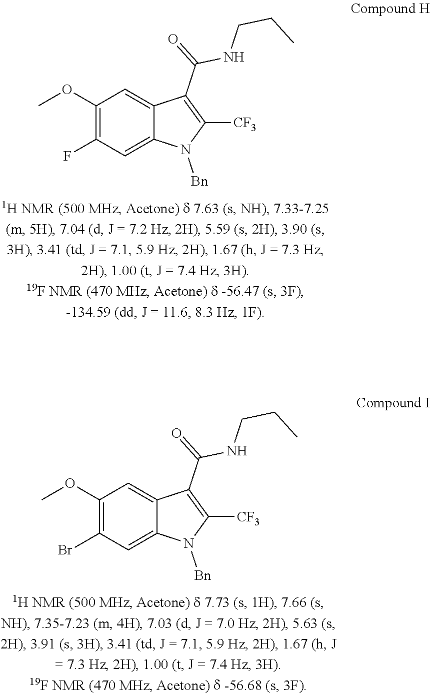

Biological Experiments

Improvement of Locomotor Function: The compounds were first assessed for their effect on viability and compounds of the invention exhibited improved adult survival when TDP-43 was expressed in motor neurons. Notably, these compounds do not affect control larvae and do not appear to affect TDP-43 levels or solubility. Compounds were further tested for their ability to improve locomotor function.

Drug treatment. Compounds were dissolved in DMSO and added to Drosophila media to a final concentration of 20 μM. Age and concentration matched DMSO in Drosophila media was used as vehicle control. Crosses were set up on drug media or DMSO controls.

Drosophila genetics. TDP-43 was expressed in motor neurons using the GAL4-UAS bipartite expression system. D42 GAL4 was used as an expression driver and UAS-TDP-43 YFP transgenics were used as responder lines.

Locomotor function. Locomotion was assessed using quantitative larval turning assays. Crosses were carried out at 25° C. and wandering third instar larvae were placed on a grape juice plate at room temperature. After a 30 second acclimation period, larvae were gently turned ventral side up. They were observed until they turned over (dorsal side up) and began making a forward motion. The time it took to complete this task was recorded for at least 30 larvae per genotype.

Statistical analyses. Kruskal-Wallis was used to calculate significance using Prism 9.0 (GraphPad). (Pvalue: *<0.05; Pvalue: **<0.01; Pvalue: ***<0.001; Pvalue: ****<0.0001; n.s.: not significant). N=30-100 larvae.

Results and Discussion

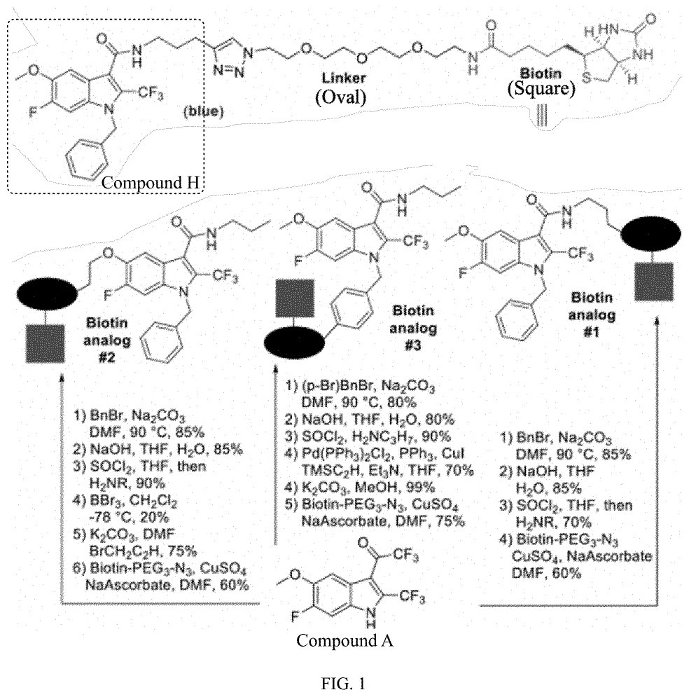

Biological test of Compound H showed a significant neuroprotective activity in Drosophila models of ALS and patient derived motor neurons. To further study the structure activity relationship (“SAR”), various biotinylated analogs of Compound H were prepared. See FIG. 1. Briefly, as shown in FIG. 1, biotin was attached to different areas of Compound H to study effects of substituents at a given position. As can be seen, biotin was attached to Compound H using a linker, e.g., via a -PEG₃-triazolyl moiety. These biotinylated Compound H analogs were tested in Drosophila models of ALS using larval turning assays to evaluate their activity in vivo. Compound H has a similar structure as Compound A but it showed improvement in locomotor function in the context of TDP-43 WT by 47% compared to 23% by Compound A. Accordingly, Compound H and its derivatives and/or analogs are superior in regards to their neuroprotective activity.

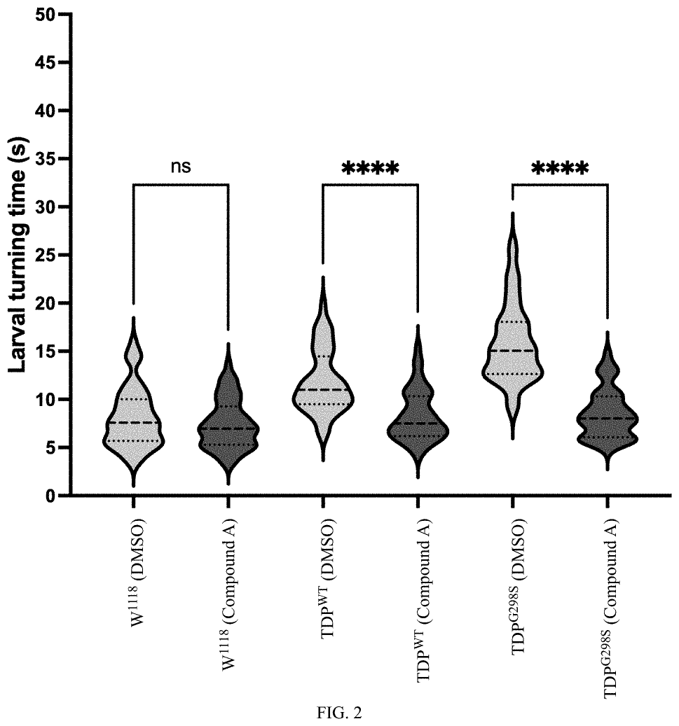

Compound A mitigates TDP-43 dependent locomotor defects: As shown in FIG. 2, Compound A significantly improved locomotor function in Drosophila models of ALS based on TDP-43 proteinopathy. In particular, 20 μM of Compound A improved locomotor function in larvae expressing either TDP-43^{WT} of TDP-43^{G298S} in motor neurons. Kruskal Wallis was used to evaluate statistical significance (Pvalue: ****<0.0001; ns: not significant. N=30-100 larvae/genotype).

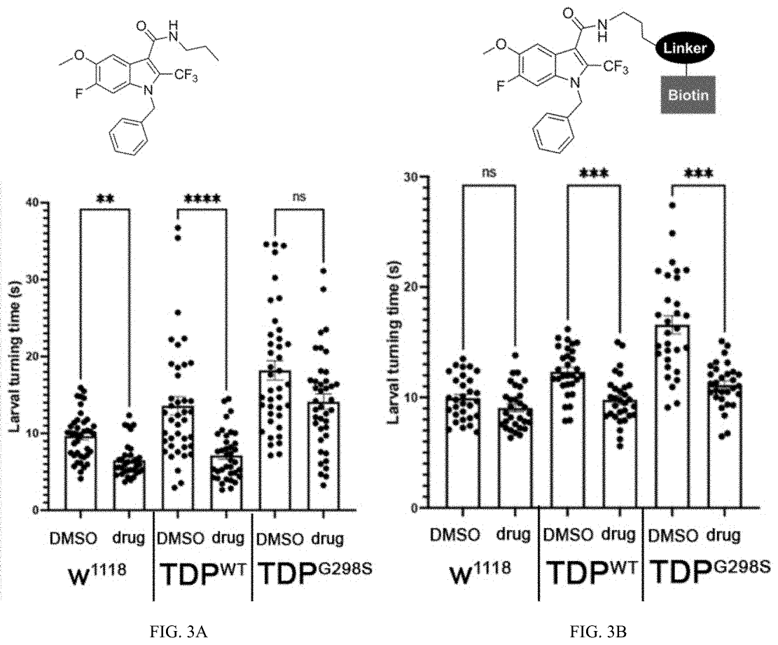

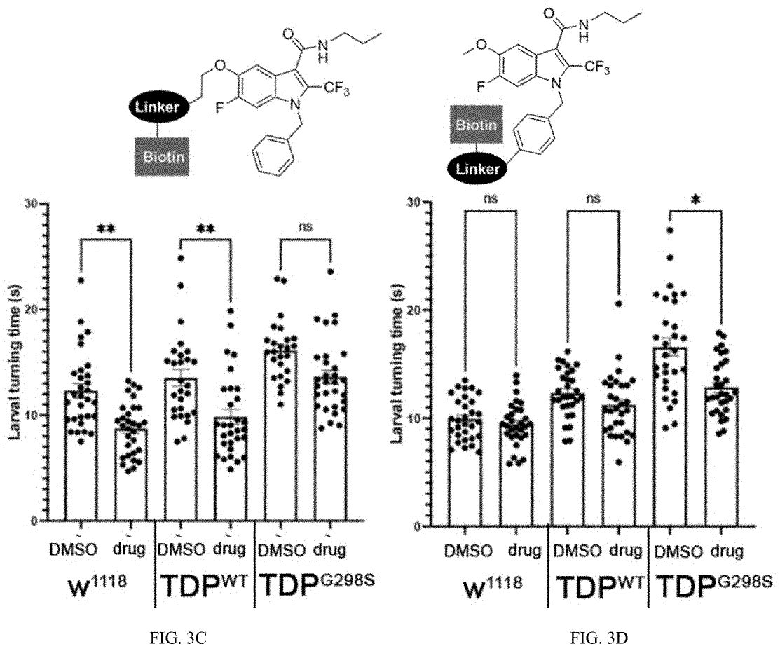

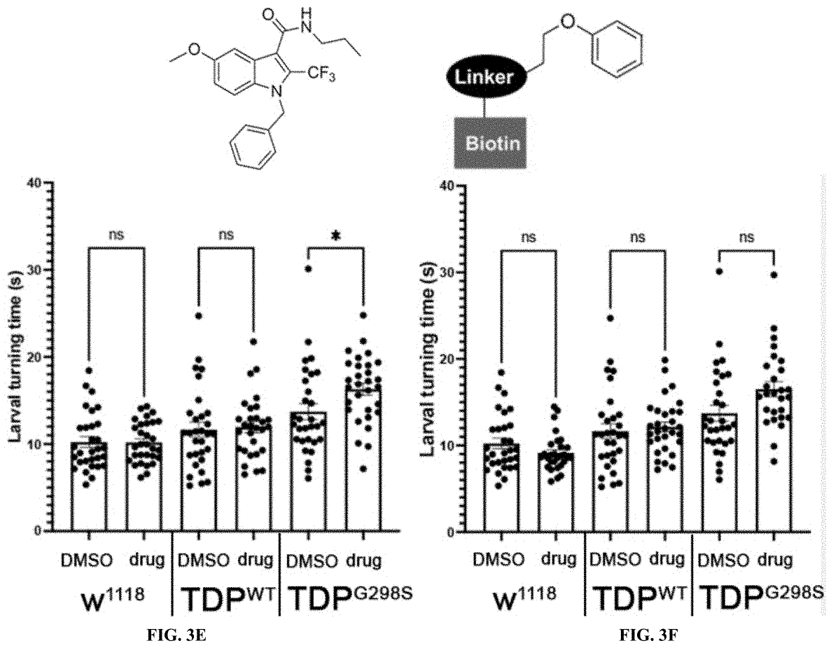

Biotinylated Compound H: Various biotinylated analogs of Compound H were tested in Drosophila models of ALS using larval turning assays to evaluate their activity in vivo. Results are shown in FIGS. 3A-3F. In particular, in FIGS. 3A-3F biotinylated chemical structures of Compound H analogs are shown on the top of the figures, and the results of locomotor function assays are shown on the bottom portion of the figures. Results show specific differential effects on locomotor activity in flies. Genotypes and treatments (DMSO versus drug) are shown in the graph. N=>30 larvae per genotype and treatment. Plots show means and SEMs. Kruskal-Wallis was used to calculate significance. (Pvalue: *<0.05; Pvalue: **<0.01; Pvalue: ***<0.001; Pvalue: ****<0.0001; n.s.: not significant).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for treating a clinical condition associated with a neurodegenerative disease or locomotor dysfunction in a subject, said method comprising administering to the subject in need of such a treatment a therapeutically effective amount of a compound of the formula: 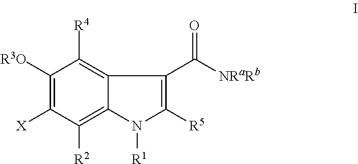
or a pharmaceutically acceptable salt thereof, or a combination thereof,
thereby treating the clinical condition associated with locomotor dysfunction in said subject,
wherein
R¹ is hydrogen, alkyl, optionally substituted aralkyl, optionally substituted aryl, or a nitrogen protecting group;
each of R² and R⁴ is independently hydrogen, alkyl, haloalkyl, halide, vinyl, alkynyl, —CHO, —C(═O)R^{c} (ketone), —CO₂R^{d} (ester), —OR^{e}, —OSO₂R^{f}, optionally substituted aryl and optionally substituted heteroaryl, wherein each of R^{c}, R^{d}, R^{e}, and R^{f} is independently alkyl, aryl, or aralkyl;
R³ is alkyl or haloalkyl;
R⁵ is alkyl or haloalkyl;
each of R^{a} and R^{b} is independently hydrogen or alkyl; and
X is halide, CN, OR^{x1}, NR^{x2}R^{x3} wherein each of R^{x1}, R^{x2}, R^{x3} are independently hydrogen, alkyl, cycloalkyl, optionally substituted aralkyl, optionally substituted aryl.

2. The method of claim 1, wherein R¹ is optionally substituted aralkyl.

3. The method of claim 2, wherein R¹ is optionally substituted benzyl.

4. The method of claim 1, wherein R² and R⁴ are hydrogen.

5. The method of claim 1, wherein R³ is C₁-C₆ alkyl.

6. The method of claim 5, wherein R³ is methyl or ethyl.

7. The method of claim 1, wherein X is halide.

8. The method of claim 7, wherein X is F or Cl.

9. The method of claim 1, wherein at least one of R^{a} or R^{b} is hydrogen.

10. The method of claim 1, wherein R^{b} is alkyl.

11. The method of claim 1, wherein said neurodegenerative disease is selected from the group consisting of Alzheimer's disease, frontotemporal dementia, a frontotemporal dementia caused by mutations in progranulin protein, amyotrophic lateral sclerosis (ALS), Huntington's chorea, Creutzfeld-Jacob disease, trinucleotide repeat diseases, cerebral degenerative diseases presenile dementia, senile dementia, Parkinsonism linked to chromosome 17 (FTDP-17), progressive supranuclear palsy (PSP), Huntington's disease (HD), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarct, cerebral trauma, chronic traumatic encephalopathy, transient ischemic attack, encephalopathy, traumatic brain injury (TBI), and any combination thereof.

12. The method of claim 1, wherein said clinical condition comprises Amyotrophic Lateral Sclerosis (ALS), frontotemporal degeneration (FTD), Alzheimer's Disease, encephalopathy, or traumatic brain injury (TBI).

13. The method of claim 12, wherein said FTD comprises frontotemporal lobar degeneration with ubiquitinated inclusions.

14. The method of claim 12, wherein said treatment comprises ameliorating a symptom associated with ALS.

15. The method of claim 1 further comprising administering a compound that decreases release of glutamate to the subject.

16. The method of claim 15, wherein said compound that decreases the release of glutamate comprises riluzole.

17. The method of claim 1 further comprising administering to said subject a compound comprising baclofen, trihexyphenidyl hydrochloride, morphine sulfate, lorazepam, glycopyrrolate, benztropine mesylate, gabapentin, diazepam, tizanidine, phenytoin sodium, amitriptyline hydrochloride, benztropine mesylate, ropinirole, glycopyrrolate, glycopyrrolate, Atropine sulphate, fluvoxamine maleate, dantrolene sodium, phenytoin sodium, gabapentin, morphine sulfate, dexpramipexole, or a combination of two or more compounds thereof.

18. A compound of the formula: 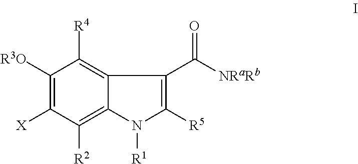
or a pharmaceutically acceptable salt thereof, or a combination thereof,
thereby treating the clinical condition associated with locomotor dysfunction in said subject,
wherein
R¹ is hydrogen, alkyl, optionally substituted aralkyl, optionally substituted aryl, or a nitrogen protecting group;
each of R² and R⁴ is independently hydrogen, alkyl, haloalkyl, halide, vinyl, alkynyl, —CHO, —C(═O)R^{c} (ketone), —CO₂R^{d} (ester), —OR^{e}, —OSO₂R^{f}, optionally substituted aryl and optionally substituted heteroaryl, wherein each of R^{c}, R^{d}, R^{e}, and R^{f} is independently alkyl or aryl;
R³ is alkyl or haloalkyl;
R⁵ is alkyl or haloalkyl;
each of R^{a} and R^{b} is independently hydrogen or alkyl; and
X is halide, CN, OR^{x1}, NR^{x2}R^{x3} wherein each of R^{x1}, R^{x2}, R^{x3} are independently hydrogen, alkyl, cycloalkyl, optionally substituted aralkyl, optionally substituted aryl.

19. The compound of claim 18, wherein said compound is of the formula: 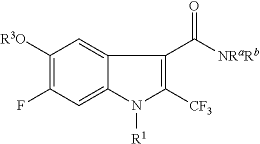
wherein R¹, R³, R^{a}, and R^{b} are those defined in claim 18.

20. A composition comprising a compound of claim 18 and a pharmaceutically acceptable excipient.
